SEQUENCE LISTING (2 sequences)

SEQ ID NO: 1 - DNA, 2117 bp, homo sapiens
ggcgcacagacgggctccgggagcccctcccgaggccccgcgcagcgcgccccgcaccctgcgccccgcgccctgcgggagggctgagccaagactccaggcgggcaggtgcggagcgagcagaggggatcacggccaagggtaggagccagtcctgcggggagagaggcgctgctgctccagctgctgctgcctccgccgccgccaccaccgagccggcgaccagagtcgggctggcaggccgggcgcgaagcggcaaggggagcgaggggtgcgctcatggagcacacgcacgcccacctcgcagccaacagctcgctgtcttggtggtcccccggctcggcctgcggcttgggtttcgtgcccgtggtctactacagcctcttgctgtgcctcggtttaccagcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtgcacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccgtgattccataggtgtggcaactactgcctctgtctaatccatttccagatgggaaggtgtcccatcctatggctgagcagctctccttaagagtgctaatccgatttcctgtctcccgcagactgggcaattctcagactggtagatgagaagagatggaagagaagaaaggagagcatgaagcttgtttttacttatgcatttatttccacagagtcgtaatgacagcaaaagctcctaccagtttgaagatgccattggagcttgtgtcatcatcctgtgaccagttaggacacaaagtagagaagtagtctgtgatttcgccctggtaccatccacagtcactgggaacccttcatttatgggacttaccaagccccagtagcacatagctgagcctgcactcttcttccgagagctgaggtcattcatcacttccctctgctgttcccaggagctaacaataatgactatttcaggatttttttcaaggtgccctttgtcctagagagggttgtggtcttgaattggctctggcactcctagcttcagaatgacactgtgggaatagaagagtattggatcccatccaaactgtggccagagcttcttcaggaaatctccaaacccgcatagctgtgacctcaaacctggggtctaaaaggcagttttctatttatcattatgtatagattttctctatctcctccaaaacaaagaccctgcctggtgcgcagggggaaaggaggaattctcgagcccagaaaaacaaaaaaata

SEQ ID NO: 2 - protein, 353 aa, homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKVSP

(19) United States
(12) Patent Application Publication
(10) Pub. No.: US 2004/0161799 A1
(43) Pub. Date: Aug. 19, 2004

(54) KOR3like-proteins and methods of modulating KOR3L-mediated activity

(76) Inventors: Andrew J. Murphy, Croton-on-Hudson, NY (US); Susan Croll-Kalish, Tarrytown, NY (US)

Correspondence Address: REGENERON PHARMACEUTICALS, INC, 777 OLD SAW MILL RIVER ROAD, TARRYTOWN, NY, 10591, US

(21) Appl. No.: 10/779,104

(22) Filed: Feb. 13, 2004

Related U.S. Application Data
(60) Provisional application No. 60/447,447, filed on Feb. 14, 2003.
(60) Provisional application No. 60/495,577, filed on Aug. 14, 2003.

(51) Int. Cl. A61K 48/00; C12Q 1/68; G01N 33/53; A61K 39/395

(52) U.S. Cl. 435/7.1; 424/146.1; 514/44; 435/6

(57) ABSTRACT

A nucleic acid encoding the GPCR protein KOR3L is described, as well as methods for screening for agents capable of modulating KOR3L related activity and treating KOR3L-mediated conditions. More specifically, methods are provided for identifying agents capable of treating KOR3L-mediated loss of balance and sensorimotor integration.

### CROSS-REFERENCE TO RELATED APPLICATIONS

This application claims the benefit under 35 USC § 119(e) of U.S. Provisional 60/447,447 filed 14 Feb. 2003 and 60/495,577 filed 14 Aug. 2003, which applications are herein specifically incorporated by reference in their entirety.

### REFERENCE TO SEQUENCE LISTING

This application refers to sequences listed in a Sequence Listing hereinto attached, which is considered to be part of the disclosure of the invention.

### BACKGROUND

1. Field of the Invention

This invention is related to KOR3-like nucleic acids and polypeptides, including assay methods, therapeutic methods, and transgenic and knock-out animals.

2. Description of Related Art

G-protein coupled receptors (GPCRs) are a class of integral membrane proteins which contain seven hydrophobic transmembrane domains that span the cell membrane and form a cluster of anti-parallel alpha helices. These receptors have been pursued as therapeutic targets for a variety of human diseases, see for example, WO 01/954582, WO 02/48358, WO 02/42461, WO 02/77001, GB 2365009, and WO 03/027142.

### BRIEF SUMMARY OF THE INVENTION

A GPCR protein, designated KOR3L (SEQ ID NO:1) and the nucleic acid which encodes it (SEQ ID NO:2), are described herein. This protein is believed to function in the mediation of locomotor activity and in the regulation of body fat, lean body mass and bone mineral density. The discovery of this protein allows for screening and therapeutic methods leading to the development of novel therapeutics useful for modulating these activities.

Accordingly, in a first aspect, the invention provides for a nucleic acid encoding KOR3L protein. More specifically, the invention features an isolated nucleic acid encoding a protein having the sequence of SEQ ID NO:2, as well as variants, derivatives, and fragments thereof.

In a second aspect, methods are provided that may be used for screening for agents capable of binding a human KOR3L protein or protein fragment having KOR3L activity. More specifically, the invention provides methods of identifying agents capable of modulating (e.g., enhancing or inhibiting) human KOR3L-mediated activity. The screening methods of the invention include in vitro and in vivo assays. Agents capable of modulating KOR3L-mediated activity preferably include agents capable of enhancing KOR3L-mediated locomotor activity, as well as agents capable of regulating body fat, lean body mass and bone mineral density.

In one embodiment of an in vitro screening method of the invention, agents capable of binding the KOR3L protein or protein fragment are identified in a cell-based assay system. More specifically, cells expressing a KOR3L protein or a protein fragment having KOR3L activity, are contacted with a test compound or a control compound, and the ability of the candidate compound to bind KOR3L or a fragment thereof is determined.

In another embodiment, agents capable of binding a KOR3L protein or protein fragment are identified in a cell-free assay system. More specifically, a native or recombinant human KOR3L protein or protein fragment is contacted with a candidate compound or a control compound, and the ability of the candidate compound to bind KOR3L or a fragment thereof is determined.

In another embodiment, agents capable of binding KOR3L or a fragment thereof are identified in vivo in an animal system. More specifically, a candidate agent or a control compound is administered to a suitable animal, and the effect on KOR3L-mediated locomotor activity, and/or regulation of body fat, lean body mass or bone mineral density is determined. Any suitable assay known to the art for determination of these activities, for example, those described in the examples below, may be used.

In a third related aspect, the invention provides methods for identifying agents capable of inhibiting the activity of human KOR3L. More specifically, the invention provides methods of identifying agents which block or inhibit activation of KOR3L, e.g., are capable of regulating body fat, lean body mass or bone mineral density that is mediated through KOR3L. In one embodiment, the agent capable of inhibiting KOR3L-mediated activity is an antagonist to a natural KOR3L ligand capable of binding to human KOR3L. In a more specific embodiment, the antagonist is an antibody. Inhibitors of KOR3L expression or activity are encompassed by the invention, including an antisense molecule capable of hybridizing with one or more nucleic acids encoding KOR3L, a ribozyme, a triple helix molecule, and a short interfering RNA (siRNA) capable of silencing KOR3L gene expression.

In a forth aspect, the invention features a method of treating a KOR3L-mediated condition, comprising administering an agent capable of inhibiting KOR3L. In one embodiment, the agent administered is a compound identified through a screening method of the invention.

In a related fifth aspect, the invention features a therapeutic method for increasing the amount of lean body mass and bone mineral density, and/or decreasing body fat, comprising administering an agent capable of inhibiting KOR3L regulation of these activities

In a sixth aspect, the invention features a method of treating a KOR3L-mediated condition, comprising administering an agent capable of activating (agonizing) KOR3L. In one embodiment, the agent administered is a compound identified through the screening method of the invention.

In a related seventh aspect, the invention features a therapeutic method for treating loss of balance or sensorimotor integration, comprising administering a therapeutically effective amount of an agent capable of activating KOR3L. In one embodiment, the agent is an activator of KOR3L identified by the screening assay of the invention. In a more specific embodiment, the agonist is an antibody. The antibody may be polyclonal, monoclonal, chimeric, humanized, or a wholly human antibody or binding portion thereof.

In an eighth aspect, the invention features pharmaceutical compositions useful for treatment of KOR3L-mediated locomotor activity, and/or regulation of body fat, lean body mass or bone mineral density. In one embodiment, the agent is identified by a screening method of the invention.

In a ninth aspect, the invention features a transgenic animal comprising a modification of an endogenous KOR3L gene. As described more fully in U.S. Pat. No. 6,856,251, the transgenic animal of the invention is generated by targeting the endogenous KOR3L gene with a large targeting vector (LTVEC). In one embodiment of the transgenic animal of the invention, the animal is a knock-out wherein the KOR3L gene is altered or deleted such that the function of the endogenous KOR3L protein is reduced or ablated. In another embodiment, the transgenic animal is a knock-in animal modified to comprise an exogenous gene. Such transgenic animals are useful, for example, in identifying agents specifically inhibiting activities that are mediated by the human KOR3L protein.

Other objects and advantages will become apparent from a review of the ensuing detailed description.

### BRIEF DESCRIPTION OF THE FIGURES

FIG. 1 shows runway gait analysis in KOR3-like knock-outs (KO) and wild type (wt) littermates.

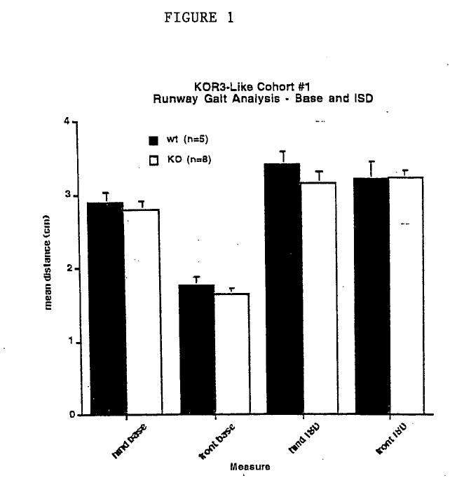

FIG. 2 shows runway gait analysis for KOR3-like KO and wt littermates.

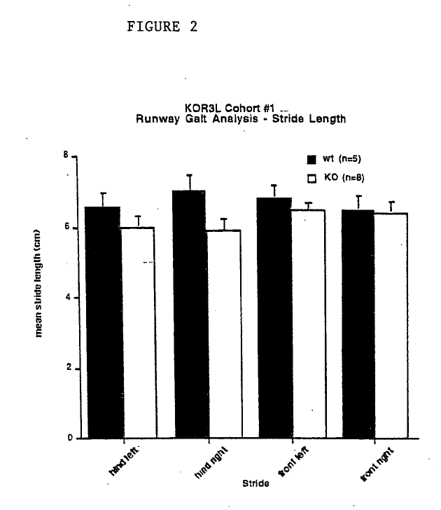

FIG. 3A-B shows open field analysis for KOR3-like KO and wt littermates.

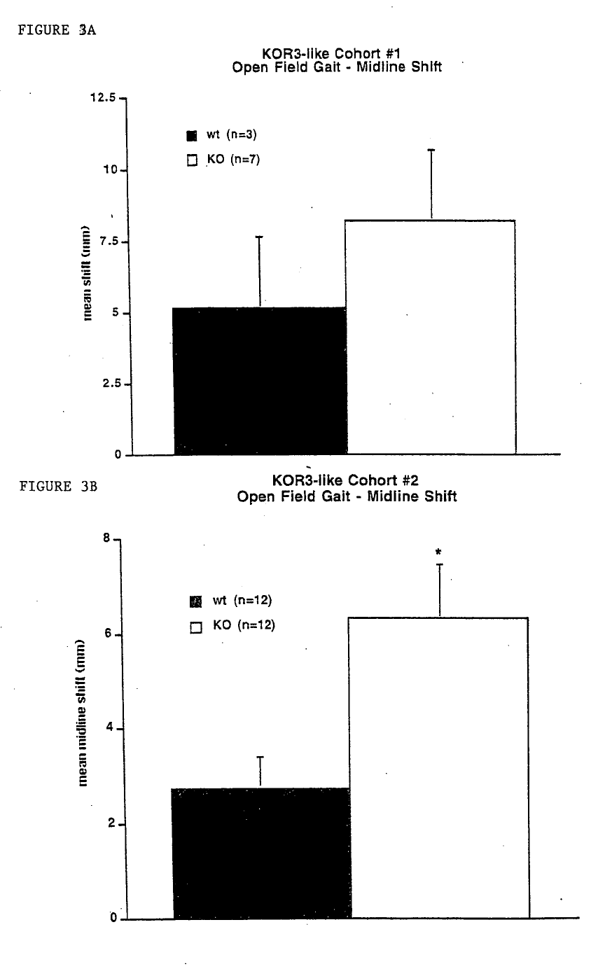

FIG. 4 shows the results of the rotorod test on wt and KOR3-like null mutants.

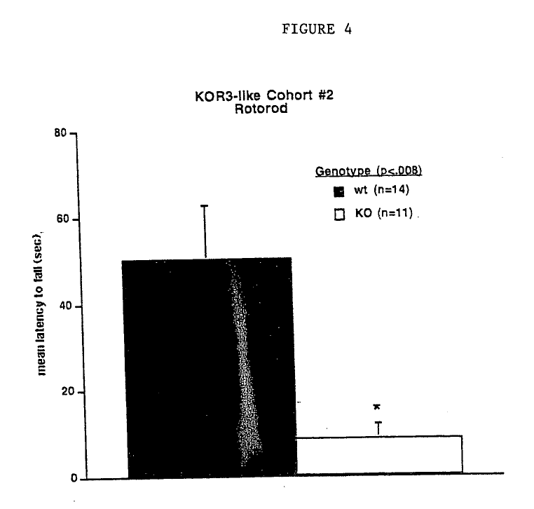

FIG. 5 shows the results of the balance beam test on wt and KOR3-like KO mice.

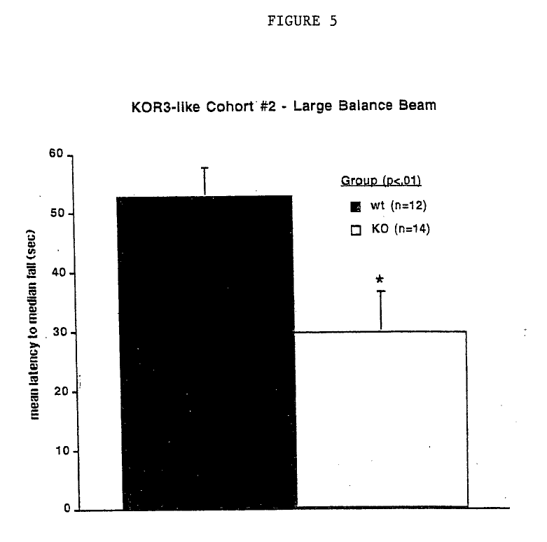

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference.

Definitions

By the term “KOR3L-mediated condition” is meant a condition which involves activation, or lack thereof, of the KOR3L protein. For example, increased lean body mass, decreased percent body fat and increased bone mineral density in KOR3L knock-outs suggests the role of KOR3L in increased adiposity, a “KOR3L mediated condition” as used herein, which would be treatable using a KOR3L inhibitor. Further, a KOR3L-mediated condition would include one in which the KOR3L receptor is insufficiently activated, thus causing, for example, loss of balance or sensorimotor integration. Such a condition would be treatable using a KOR3L agonist as described herein.

By the term “inhibitor” is meant a substance which retards or prevents a chemical or physiological reaction or response. Common inhibitors include but are not limited to antisense molecules, antibodies, antagonists and their derivatives.

A transgenic “knock-in” animal is an animal generated from a mammalian cell which carries a genetic modification resulting from the insertion of a DNA construct targeted to a predetermined, specific chromosomal location which does not alter the function and/or expression of the gene at the site of the targeted chromosomi location. A “knock-out” animal is an animal generated from a mammalian cell which carries a genetic modification resulting from the insertion of a DNA construct targeted to a predetermined, specific chromosomal location which alters the function and/or expression of a gene that was at the site of the targeted chromosomal location. In both cases, the DNA construct may encode a reporter protein such as lacZ, protein tags, and proteins, including recombinases such as Cre and FLP.

General Description

This invention is based in part on elucidation of the coding sequence and function of the human receptor designated herein as kappa opioid receptor-3like (KOR3L). The experiments described below identify the function of KOR3L as involved in the modulation of locomotor activity, and/or regulation of body fat, lean body mass or bone mineral density. Accordingly, these discoveries provide new methods for the treatment of KOR3L-mediated conditions, such as loss of balance or sensorimotor integration, by treatment with KOR3L ligands or other agonists, including, but not limited to, activating antibodies. Further, the invention provides screening assays for identification of molecules capable of inhibiting KOR3L-mediated activity, e.g., for treatment of increased adiposity.

Protein and Nucleic Acid Sequence

The nucleic acid sequence of KOR3L is shown in SEQ ID NO: 1, and the encoded amino acid sequence in SEQ ID NO: 2. The invention further encompasses nucleotide sequences that hybridize under stringent conditions to the complement of the nucleotide sequence of SEQ ID NO:1, or a fragment thereof and which encode KOR3L, wherein said stringent conditions are 30% formamide in 5×SSPE (0.18 M NaCl, 0.01 M NaPO₄, pH 7.7, 0.001 M EDTA) buffer at a temperature of 42° C. and remaining bound when subject to washing at 42° C. with 0.2×SSPE. The invention further provides for nucleotide sequences which, as a result of the degeneracy of the genetic code, differ from the nucleic acid of SEQ ID NO:1 or sequences which hybridize thereto and which encode KOR3L.

In addition, the invention contemplates vectors which comprise KOR3L encoding sequences, wherein the nucleic acid molecule is operatively linked to an expression control sequence capable of directing its expression in a host cell. The invention further contemplates host-vector systems for the production of KOR3L, including bacterial, yeast, insect, amphibian or mammalian cells.

Screening Assays

The present invention provides methods for identifying agents (e.g., candidate compounds or test compounds) that are capable of modulating (e.g., upregulating or downregulating) KOR3L-mediated activity. Agents identified through the screening method of the invention that are KOR3L agonists are potential therapeutics for use in treating KOR3L-mediated conditions involved loss of balance or sensorimotor integration. In addition, the invention provides for the identification of agents that are capable of inhibiting KOR3L-mediated regulation of body fat, lean body mass, and bone mineral density. Agents identified through the screening method of the invention are potential therapeutics for use in decreasing body fat, and/or increasing lean body mass or bone mineral density.

Examples of agents include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art. Test compounds further include, for example, antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies). Further, agents or libraries of compounds may be presented, for example, in solution, on beads, chips, bacteria, spores, plasmids or phage.

In one embodiment, agents that bind KOR3L are identified in a cell-based assay system. In accordance with this embodiment, cells expressing a KOR3L protein or protein fragment are contacted with a candidate (or a control compound), and the ability of the candidate compound to bind KOR3L is determined. The cell may be of prokaryotic origin (e.g., E. coli) or eukaryotic origin (e.g., yeast or mammalian). In specific embodiments, the cell is a KOR3L expressing mammalian cell, such as, for example, a COS-7 cell, a 293 human embryonic kidney cell, a NIH 3T3 cell, or Chinese hamster ovary (CHO) cell. Further, the cells may express a KOR3L protein or protein fragment endogenously or be genetically engineered to express a KOR3L protein or protein fragment. To identify ligands of KOR3L, cells expressing the receptor may be screened against a panel of know peptides utilizing a bioluminescent signal such as the aequorin luminescence assays (see, for example, Button et al. (1993) Cell. Calcium 14:663-671; Liu et al. (1999) Biochem. Biophys. Res. Comm. 266:174-178; Ungrin et al. (1999) Anal. Biochem. 272:34-42; Fujii et al. (2000) J. Biol. Chem 275:21086-21074; Raddatz et al. (2000) J. Biol. Chem. 275:32452-32459; and Shan et al. (2000) J. Biol. Chem. 275:39482-39486, which references are herein specifically incorporated by reference in their entireties). In these binding assays, the peptide to be tested is labeled. Cells expressing the KOR3L receptor are then incubated with labeled test compounds, in binding buffer, in cell culture dishes. To determine non-specific binding, unlabeled peptide may be added to the wells. After the incubation, bound and free peptides are separated and detection activity measured in each well.

The ability of the candidate compound to alter the activity of KOR3L can be determined by methods known to those of skill in the art, for example, by flow cytometry, a scintillation assay, immunoprecipitation or western blot analysis. For example, modulators of KOR3L-mediated conditions may be identified using a biological readout in cells expressing a KOR3L protein or protein fragment. Agonists or antagonists are identified by incubating cells or cell fragments expressing KOR3L with test compound and measuring a biological response in these cells and in parallel cells or cell fragments not expressing KOR3L. An increased biological response in the cells or cell fragments expressing KOR3L compared to the parallel cells or cell fragments indicates the presence of an agonist in the test sample, whereas a decreased biological response indicates an antagonist.

In more specific embodiments, detection of binding and/or modulation of a test agent to a KOR3L protein may be accomplished by detecting a biological response, such as, for example, measuring Ca²⁺ ion flux, cAMP, IP₃, PIP₃ and transcription of reporter genes. Suitable reporter genes include endogenous genes as well as exogenous genes that are introduced into a cell by any of the standard methods familiar to the skilled artisan, such as transfection, electroporation, lipofection and viral infection. The invention further includes other end point assays to identify compounds that modulate (stimulate or inhibit) receptor activity, such as those associated with signal transduction.

In another embodiment, agents that modulate KOR3L-mediated activity are identified in a cell-free assay system. In accordance with this embodiment, a KOR3L protein or protein fragment is contacted with a test (or control) compound and the ability of the test compound to bind KOR3L is determined. In vitro binding assays employ a mixture of components including a KOR3L protein or protein fragment, which may be part of a fusion product with another peptide or polypeptide, e.g., a tag for detection or anchoring, and a sample suspected of containing a natural KOR3L binding target. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature which facilitates optimal binding. The mixture is incubated under conditions whereby the KOR3L protein binds the test compound. Incubation periods are chosen for optimal binding but are also minimized to facilitate rapid, high-throughput screening.

After incubation, the binding between the KOR3L protein or protein fragment and the suspected binding target is detected by any convenient way. When a separation step is useful to separate bound from unbound components, separation may be effected by, for example, precipitation or immobilization, followed by washing by, e.g., membrane filtration or gel chromatography. One of the assay components may be labeled which provides for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radiative emissions, nonradiative energy transfers, or indirectly detected with antibody conjugates.

It may be desirable to immobilize either the receptor protein, or fragment, or its target molecule to facilitate separation of complexes from uncomplexed forms of one of the proteins, as well as to accommodate automation of the assay. Techniques for immobilizing proteins on matrices can be used in the drug screening assays. In one embodiment, a fusion protein is provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule, as well as enzyme-linked assays which rely on detecting an enzymatic activity associated with the target molecule.

In another embodiment, agents that modulate (i.e., upregulate or downregulate) KOR3L-mediated activity are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the KOR3L-mediated activity is determined. More specifically, this method may be used to identify an agent capable of modulating KOR3L-mediated locomotor activity, and/or regulation of body fat, lean body mass or bone mineral density.

Antibodies to Human KOR3L Protein and Ligands

The present invention provides for an antibody which specifically binds human KOR3L and is useful in the modulation of locomotor activity, and/or regulation of body fat, lean body mass or bone mineral density. According to the invention, a KOR3L protein, protein fragment, derivative or variant, may be used as an immunogen to generate immunospecific antibodies. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105 ). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the treatment of a KOR3L-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a transgenic knock-out animal having a modified endogenous KOR3L gene. A transgenic animal can be produced by introducing nucleic acid into the male pronuclei of a fertilized oocyte, e.g., by microinjection, retroviral infection, and allowing the oocyte to develop in a pseudopregnant female foster animal. Still further, the invention contemplates a transgenic animal having an exogenous KOR3L gene generated by introduction of any KOR3L-encoding nucleotide sequence which can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the KOR3L protein to particular cells.

Transgenic animals containing a modified KOR3L gene as described herein are useful to identify KOR3L function. Further, animals containing an exogenous KOR3L gene, e.g., a human KOR3L gene, may be useful in an in vivo context since various physiological factors that are present in vivo and that could effect ligand binding, KOR3L activation, and signal transduction, may not be evident from in vitro cell-free or cell-based assays. Accordingly, it is useful to provide non-human transgenic animals to assay in vivo KOR3L protein function, including ligand interaction, the effect of specific mutant KOR3L proteins on KOR3L protein function and ligand interaction, and the effect of chimeric KOR3L proteins. It is also possible to assess the effect of null mutations, that is mutations that substantially or completely eliminate one or more KOR3L protein functions.

Specific Embodiments

As described below, LacZ expression patterns in mice for which the KOR3-like gene has been replaced with LacZ show a preferential expression of KOR3-like in the motor systems and some sensory systems in brain. Specifically, KOR3-like is expressed in the lateral striatum, globus pallidus, inferior olivary complex, and deep nuclei of the cerebellum, all of which are components of the motor system. In addition, KOR3-like is expressed in some sensory structures, most notably the vestibular system and the dorsal root ganglia. Because of this expression pattern in brain motor systems, as well as sensory systems that can modulate the motor systems, experiments were conducted to ascertain the role of KOR3-like in motor functioning and somatosensation.

As described in Example 5 below, KOR3-like mutants demonstrated a significant impairment in tasks that measured balance or sensorimotor integration. Specifically, these mice showed midline shift when walking in a curve, and had significantly decreased latencies to fall off of both the rotorod and the balance beam. Given the localization of the KOR3-like gene suggested by LacZ expression patterns, this pattern of deficits is consistent with abnormalities in various motor or sensory structures.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Identification of Human KOR3L

KOR3-like was an orphan receptor identified from genomic DNA. After bioinformatics refinement, the full-length receptor was confirmed by RT-PCR and sequencing. Initial TaqMan analysis identified fetal brain, hypothalamus and pituitary as sites of expression.

### Example 2

### Expression of Human KOR3L

KOR3-like was knocked out using VelociGene technology as described in U.S. Pat. No. 6,856,251, which is incorporated in its entirety herein. LacZ staining was performed on chimeras (V'Gene GEG, and heterozygotes revealing predominantly brain expression (olfactory bulbs, habenula, Caudate-Putamen, preoptic Hypothalamus, Supraoptic Hypothalamus, Globus Pallidus, Retrochiasmatic Hypothalamus, Anterior Hypothalamus, Entopeduncular nucleus, Lateral Hypothalamus, Parafascicular Nucleus, Substantia Nigra, Cerebellar Nuclei, Vestibular Nuclei, Inferior Olivary Complex and Area Postrema) as well as expression in trigeminal nerve, anterior pituitary and dorsal root ganglia of the spinal cord. These sites of expression suggest activities of this receptor in modulations locomoter activity, olfactory or pain sensation and/or hormonal regulation.

### Example 3

### Phenotyping of KOR3L Knockouts

In the phenotyping of homozygous knock-out animals, pDEXA analysis showed the following differences in male animals (8 knock-out animals compared to 8 wild type animals derived from the same F1 crosses): increased lean body mass, decreased percent body fat and increased bone mineral density, as shown the table below:

| | Lean Content | Fat Content | Fat % |
|---|---|---|---|
| wt | 21.40938 | 4.827875 | 0.17292182 |
| ko | 25.58 | 1.93619375 | 0.06408279 |

### Example 4

### KOR3-Like Activity in Tissue Culture

Human KOR3-like cDNA was over-expressed in HEK293 cells in order to ascertain which G-protein pathway might be used to screen for modulators of its activity (agonists or antagonists). This analysis revealed that KOR3-like is able to stimulate CRE-luc (cAMP-responsive-element luciferase, a reporter responsive to stimulation of Gs) 5.4 fold, NF-AT-luc (nuclear factor of activated T cells-luciferase, a reporter responsive to Gq activation) 4.3 fold and SRE-luc (serum response factor-luciferase, a reporter responsive to several GPCRs through an undefined mechanism) 2.7 fold.

### Example 5

### The effect of KOR3-Like on Motor Activity

Materials and Methods. The mice used were male and female KOR3-like null mutants and their wild type littermates, tested at 8-20 weeks of age. Two separate cohorts of mice were tested at two different times. All subjects had food and water available ad libitum, and were maintained in a controlled temperature and humidity environment on a 12:12 light-dark cycle (lights on 0600).

Behavioral Testing. All behavioral testing was conducted by a behaviorist blind to the genotype of the animals. Behavioral testing was conducted in a quiet behavioral room within the animal facility. Animals were placed in the behavioral room for one hour before testing to ensure acclimation to the testing environment.

Gait Analysis. For analysis of gait in the mice, all mice were painted with red non-toxic paint on their forepaws and blue non-toxic paint on their hindpaws before being placed onto paper to record their walking patterns. Each mouse was tested on two separate walking tasks. Specifically, mice were placed onto paper on an open field where their walking was not constrained in any way. In addition, each mouse was placed on a runway, where they will walk from the start to finish. After the paint had dried, an experimenter blind to the genotype of the animals took measurements from a walking sample contained 4 consecutive strides, all of which could be clearly seen (i.e. no smudging or fading of footprints). Some mice needed to be placed on the apparatuses two to three times to obtain an acceptable sample. Measurements taken were stride length (length from one paw placement to the next placement of the same paw), base of support (horizontal distance, or width, between the right and left paw), inter-step distance (vertical distance, or length, between the right and left paw), and toe spread (distance between the second and third front toe—hind toe was not done because animals were toe-clipped), all of which are standard measurements. In addition, we created a novel measurement, termed “midline shift.” This measurement was created in order to allow for quantification of an abnormality noted in the gait of some animals. For midline shift, the midline point was determined for the horizontal distance (width) between the left and right hindpaw, and the left and right forepaw. The horizontal distance between the midpoints for hindpaws and forepaws was determined. In normal animals, the midline shift, or difference between the horizontal location of the two midpoints, should approach 0.

Analysis of balance and motor integration. KOR3-like animals were placed on a standard rotorod apparatus, rotating at 10 rpm, until they fell off. The maximum trial length was 2 minutes. Animals were placed onto the rotorod 3 times, and the median trial was taken as the measurement analyzed.

Balance beam. Animals were placed onto two different balance beam apparatuses to evaluate their ability to maintain balance on a beam. A thin rod, 5 mm in diameter, and a thick rod, 2 cm in diameter, were used. The rods were suspended approximately 40 cm from a soft pad. On the thin rod, animals could compensate behaviorally by wrapping their feet around the rod and hanging. On the thick rod, animals could behaviorally compensate by freezing. Therefore, both rods were used to maximize the chances of detecting an effect. Animals were placed on each rod 3 times, and the median latency to fall off the rod was analyzed as a measure of balance.

Von Frey hairs. Because motor abnormalities can occur when somatosensory input is abnormal, thereby impacting sensorimotor integration, animals were tested for somatosensory sensitivity using Von Frey hairs. Animals were placed on a screen, and an experimenter applied pressure to the animals' hindpaws as they rested on the screen. Von Frey hairs were introduced in an ascending series, and the pressure corresponding to the first hair which consistently caused foot withdrawal was recorded as the somatosensory sensitivity threshold.

Results. Gait Analysis. Gait analysis revealed no significant differences between KOR3-like knock-outs and their wild type littermates in any of the standard gait measurements. In the open field, however, KOR3-like knock-outs showed a significant midline shift, such that their forepaws were displaced laterally relative to their forepaws. This effect was not observed when animals walked in the runway apparatus (data not shown). Visual inspection of the footprints revealed that the midline shift only occurred when animals were walking in a curve or turning a corner. Midline shift was not observed in either apparatus while the animals were walking in a straight line. Therefore, it is likely that the animals cannot maintain a symmetrical posture when confronted with the challenge of a corner. These data suggest difficulties with either balance or sensorimotor integration.

Balance and motor integration. The rotorod measures both balance and sensorimotor integration in animals. The rotorod test revealed a significant impairment in the KOR3-like null mutants, such that they had difficulty remaining on the rotorod apparatus. This impairment confirms that KOR3-like mutants have deficiencies in either balance or sensorimotor integration.

Balance Beams. KOR3-like null mutants showed no significant impairment relative to wild type controls when placed on the thin balance beam (data not shown). However, most animals encircled the beam with their paws and hung, rather than balancing, on the beam. On the thicker balance beam, for which this behavioral strategy does not work, KOR3-like knock-outs were significantly impaired relative to their wild type littermates. This impairment on the thick balance beam lends further support to the assertion that balance is abnormal in these mutants.

Von Frey Hairs. KOR3-like null mutants showed no deficiency in somatosensation using the Von Frey hair paradigm. All mice tested showed similar response patterns to the hairs, without regard to genotype (data not shown).

What is claimed is:

1. A method for identifying an agent capable of modulating a KOR3L protein, or protein fragment activity, comprising:
(a) contacting a test agent with a KOR3L protein, or protein fragment; and
(b) determining the ability of the test agent to bind KOR3L protein or protein fragment.

2. A method for identifying an agent capable of modulating a KOR3L protein, or protein fragment activity, comprising:
(a) administering a test agent to an animal expressing a KOR3L protein, or protein fragment; and
(b) determining the ability of the test agent to modulate KOR3L protein or protein fragment.

3. The method of claim 2, wherein the test agent is an antibody to KOR3L.

4. The method of claim 3, wherein the antibody is an activating or blocking antibody.

5. The method of claim 2, wherein the test agent is capable of inhibiting KOR3L expression.

6. The method of claim 5, wherein the agent is selected from the group consisting of an antisense molecule capable of hybridizing with one or more nucleic acids encoding KOR3L, a ribozyme, a triple helix molecule, and a short interfering RNA (siRNA) capable of silencing KOR3L gene expression.

7. A therapeutic method for treating KOR3L-mediated loss of balance or sensorimotor integration, comprising administering a therapeutically effective amount of an agent capable of activating KOR3L.

8. The therapeutic method of claim 7, wherein the agent is an activating antibody.

9. The therapeutic method of claim 8, wherein the antibody is polyclonal, monoclonal, chimeric, humanized, or a wholly human antibody.

10. A therapeutic method for treating obesity, comprising administering a therapeutically effective amount of an agent capable of inhibiting KOR3L.

11. The method of claim 10, wherein the agent is an antagonist of KOR3L.

12. The method of claim 11, wherein the antagonism is an antibody.

13. The method of claim 10, wherein the agent is an inhibitor of KOR3L expression.

14. The method of claim 13, wherein the inhibitor is selected from the group consisting of an antisense molecule capable of hybridizing to a nucleic acid encoding KOR3L, a ribozyme, a triple helix molecule, and a short interfering RNA (siRNA) capable of silencing KOR3L gene expression.

15. A pharmaceutical composition, comprising an agent identified by a screening method of claim 2 and a pharmaceutical acceptable carrier.

16. A transgenic animal, comprising a modification of an endogenous KOR3L gene.

17. The transgenic animal of claim 16, wherein the modification is an alteration or deletion of the endogenous KOR3L gene such that the function of the endogenous KOR3L protein is reduced or ablated.

18. The transgenic animal of claim 17, further comprising a human KOR3L gene.
